(19) United States
(12) Patent Application Publication
(10) Pub. No.: US 2026/0240670 A1
(43) Pub. Date: Aug. 20, 2026

(54) PROCEDURE, INSTRUMENTS AND CRIMPER FOR CRIMPING AN IMPLANTABLE DEVICE

(71) Applicant: INNOVHEART S.r.l., Milano (MI) (IT)

(72) Inventors: Giovanni RIGHINI, Gland (CH); Enrico BRESCIANO, Milano (MI) (IT); Gianluca PANE, Ivrea (TO) (IT)

(21) Appl. No.: 19/164,315

(22) PCT Filed: Mar. 12, 2024
(86) PCT No.: PCT/IB2024/052368

§ 371 Date: Sep. 11, 2025

(30) Foreign Application Priority Data
Mar. 14, 2023 (IT) 102023000004791

(51) Int. Cl. A61F 2/95; A61F 2/24

(52) U.S. Cl. CPC A61F 2/9522; A61F 2/2418

(57) ABSTRACT

A set of instruments for crimping a support structure of a heart valve having a first crown and a second crown. The set includes at least one instrument comprising a handle terminating at one end as a cylindrical body, the cylindrical body having a lateral surface provided with seats for receiving therein the front crown of the support structure. The set further includes a cap slidable on the cylindrical body to block the first crown there, and at least one crimping ring for crimping the second crown.

### FIELD OF THE INVENTION

The present invention relates to a procedure, a set of instruments and a crimper for crimping an implantable device.

The invention has been developed with particular, though non-limiting, regard to crimping a support structure of a heart valve prosthesis, and particularly the support structure of a valve prosthesis for replacing an atrioventricular valve. The term “support structure” is intended here and below to be understood to be the deformable structure to which there are fixed the prosthetic leaflets which, in the implanted valve prosthesis, control the blood flow.

### TECHNOLOGICAL BACKGROUND

There are known a number of crimping devices for prosthetic heart valves. The term “crimping” is intended to be understood in the field to be the operation of reducing the dimensions, usually radial dimensions, of an implantable device and in particular a vascular stent or a valve prosthesis. This operation is normally necessary in order to mount the implantable device in catheters or other delivery systems which have a low profile and which are used for implantation in a patient by means of minimally invasive procedures. The action of crimping may involve the entire implantable device or only a portion thereof.

The implantable devices to be subjected to the crimping operation may be long, that is to say with an axial length which is significantly greater than the diameter thereof in an expanded configuration, or short, that is to say characterized by an axial length smaller than the diameter.

Furthermore, the devices can be shaped, that is to say have different diameters in correspondence with different sections or also have a geometry different from the cylindrical geometry, with complex three-dimensional profiles. The need for mechanically reducing the radial dimension of structures having geometries which are in any case complex, as required by the transcatheter procedures, avoiding risks of damaging the implantable device itself, requires the development of crimping devices and procedures which are particularly versatile and efficient. Furthermore, such crimping devices also have to be easy and safe to use, as the crimping procedure, particularly of prosthetic valve devices, is carried out directly in the operating theatre, in a sterile environment, immediately before the implantation procedure, by theatre staff who are suitably trained but not specialized or with particular technical competences.

The patent EP2014257 is directed towards a crimping device which is specific to short prostheses. Although the device described therein is capable of crimping a number of short devices, it is not suitable for complex geometries and in particular it is not suitable if high precision is required at the point of application of the crimping action.

More generally, a problem of the devices and procedures of the known type is that they cannot ensure a high level of precision of the plane in which the crimping takes place and in any case they do not allow the crimping of a valve with a non-constant section or complex geometry without the risk of causing the collapse thereof.

### STATEMENT OF INVENTION

An object of the invention is to solve the problems of the prior art. Another object is to provide a procedure and a set of instruments and a crimper which are particularly suitable for crimping a support structure of an implantable device with a non-constant section in a reliable and reproducible manner. Another object is to provide economic, simple, reliable devices to use which are capable of completing the crimping operation within acceptable times compatible with the duration of the implantation procedure.

In order to achieve these objects, the invention relates to a procedure, a set of instruments and a crimper as set out by the appended claims.

According to a first aspect, there are described crimping procedures and related sets of devices which provide for a succession of steps with intermediate configurations. The procedures and the sets of devices minimize the non-uniformity of the deformation and therefore the state of stress inside the structure of the prosthesis. The subdivision of the crimping procedure into successive steps also increases the simplicity and reproducibility of compacting the implantable device, to the benefit of safety and rapidity of execution.

According to a second aspect, there is described a procedure for crimping the support structure of an implantable device, wherein the structure has two ends. The procedure may comprise a first step, in which one of the two ends of the support structure is crimped, and a second step, in which the other end is crimped. The separate crimping of the two ends introduces a gradual nature to the operation which can thereby be carried out with greater precision and safety, avoiding irregular or uncontrolled deformations. Preferably, the steps are repeated at least one additional time, preferably in the same order, to bring the implantable device to a final, desired crimping diameter. The crimping action is thereby even more gradual and has a minimal risk of valve collapse during the crimping. Even more preferably, the above-described steps are repeated a third time.

During the final repetition, it is possible to mount the support structure on a catheter and/or a delivery system for the subsequent implantation of the prosthesis. The final step can be carried out just before the implantation while the previous steps can be carried out adequately in advance with respect to the time of use of the delivery system.

This procedure which is subdivided into a plurality of steps therefore also allows more effective synchronization of the crimping times with the times of the implantation procedure, making the prosthesis mounted on the delivery system available at the time of its use, but at the same time avoiding the risks of having the prosthesis stay longer than necessary in its configuration of maximum compaction. In the case, for example, of a transcatheter valve prosthesis, it is in fact advantageous to be able to carry out a first part of the crimping adequately in advance with respect to the exact time of the implantation, achieving an intermediate crimping configuration which on one hand avoids excessive compression of the biological assembly which usually constitutes the functional component of the prosthesis, but at the same time allows a rapid and safe completion of the crimping procedure. In this manner, the final crimping step can be well-synchronized with the times of the implantation procedure, avoiding an excessively long period of time for the valve prosthesis to remain in the completely crimped configuration, which could be detrimental to the functional component of the prosthesis, and at the same time making the delivery system promptly available for the operator at the time suitable for implantation, avoiding any increase in duration of the intervention procedure.

According to another aspect, there is described a procedure for crimping the support structure of an implantable device, wherein the procedure may comprise a first step, in which there is crimped one of the two ends of the support structure, which can be carried out with a crimper with a suitable design and an instrument comprising a cylindrical body having projections at an external lateral surface thereof.

According to an aspect, there is described a procedure for crimping the support structure of an implantable device, wherein the procedure may comprise a first step, in which there is crimped one of the two ends of the support structure, and a second step, in which the other end is crimped, which can be carried out with an instrument comprising a crimping ring.

According to an aspect, there is described a procedure for crimping the support structure of an atrioventricular heart valve prosthesis, wherein the support structure has an atrial crown and a ventricular crown. The procedure may comprise a first step, in which the atrial crown is crimped, and a second step, in which the ventricular crown is crimped, or vice versa.

According to an aspect, there is described a procedure for crimping the support structure of an atrioventricular heart valve prosthesis which is subdivided into a plurality of steps, comprising a final step which may provide for the insertion of the crimped valve prosthesis in a valve capsule of a delivery system.

According to another aspect, there is also described a set of instruments for crimping the support structure of an atrioventricular heart valve prosthesis, comprising an atrial crown and a ventricular crown. The set may comprise at least one instrument of a first type. An instrument of a first type may comprise a cylindrical body with a diameter which is preferably smaller than the atrial crown of the support structure, preferably fixedly joined to a handle; there can be provided seats on the lateral surface of the cylinder for receiving therein and preferably anchoring therein the atrial crown. The instrument of a first type may comprise an atrial cap, which can slide on the cylindrical body, in order to block the atrial crown on the cylindrical body. The atrial cap therefore has the function of fixing the diameter of the atrial crown at a diameter which is preferably smaller than the diameter of the support structure of a non-deformed heart valve. The set may comprise at least one instrument of a second type. An instrument of a second type may comprise a crimping ring in order to crimp the ventricular crown of the support structure.

A set of instruments may also comprise a second crimping ring for further crimping the ventricular crown and a positioning Cap in order to assist in an insertion operation of the second crimping ring on the ventricular portion of the valve prosthesis.

Preferably, the set instruments comprises a first instrument and a second instrument of the first type, the second instrument differing from the first instrument as a result of the reduced radial dimensions of the cylindrical body and the atrial cap. Preferably, the set of instruments also comprises three different instruments of the second type, differing mainly as a result of the internal diameter of the crimping ring. Preferably, at least one instrument of the second type also comprises a positioning cap, preferably associated with the crimping ring with a smaller diameter. It is naturally also possible to have different compositions of the set of instruments for crimping support structures of implantable devices.

According to another aspect, there is described a procedure for crimping a support structure of an implantable device, for example, an atrioventricular heart valve prosthesis, by combining the use of a set of instruments for crimping with a crimper of a suitable design.

According to another aspect, there is described a crimper for crimping a support structure of an implantable device, which is configured to reduce the radial dimension of the support structure in a manner limited to a length portion thereof which is negligible with respect to the overall length of the support structure itself. The crimper comprises a plurality of movable crimping structures, preferably with a thickness which is significantly reduced with respect to the axial dimension of the support structure, which collectively define a plane with a hole. The hole has a diameter which may vary as desired and which is controllable by acting on the crimping structures. The crimping structures can further define a pair of apertures.

### BRIEF DESCRIPTION OF THE DRAWINGS

Additional features and advantages will become evident from the following detailed description of a preferred embodiment of the invention with reference to the appended drawings which are provided merely by way of non-limiting example and in which:

FIG. 1 is a perspective view of a crimper and a support structure of an atrioventricular heart valve prosthesis to be crimped,

FIG. 2 is a side view of the crimper of FIG. 1 in a configuration with maximum radial crimping action,

FIG. 3 shows a first pair of instruments belonging to a set of instruments for crimping,

FIG. 4 shows a second pair of instruments belonging to a set of instruments for crimping,

FIG. 5 shows a third instrument belonging to a set of instruments for crimping,

FIG. 6 shows in detail the support structure of an atrioventricular heart valve prosthesis in a first crimping step,

FIG. 7 shows the support structure of an atrioventricular heart valve prosthesis with the atrial crown and the ventricular crown crimped with the first pair of instruments,

FIG. 8 shows the support structure of an atrioventricular heart valve prosthesis inserted in the crimper, with the atrial crown crimped on a second instrument,

FIG. 9 shows the support structure of an atrioventricular heart valve prosthesis with the atrial crown and the ventricular crown crimped with the second pair of instruments,

FIGS. 10 to 16 show steps following the step of FIG. 9 for inserting the prosthesis in a valve capsule of a release instrument of the valve with retrograde access to the native valve, in particular:

FIG. 10 shows the support structure of an atrioventricular heart valve prosthesis with the ventricular crown crimped by a ring of the second pair of instruments and the insertion between the prosthetic leaflets of an empty valve capsule;

FIG. 11 shows the support structure of an atrioventricular heart valve prosthesis with the atrial crown crimped on the delivery system of FIG. 10,

FIG. 12 shows the support structure of an atrioventricular heart valve prosthesis with the atrial crown inserted in the valve capsule of FIG. 10,

FIG. 13 shows the support structure of an atrioventricular heart valve prosthesis with the atrial crown inserted in the valve capsule of FIG. 10 and the ventricular crown crimped by the crimper,

FIG. 14 shows the support structure of an atrioventricular heart valve prosthesis with the atrial crown inserted in the valve capsule of FIG. 10 and with the ventricular crown partially crimped by a third instrument,

FIG. 15 shows the support structure Of an atrioventricular heart valve prosthesis with the ventricular crown completely crimped by the third instrument using the positioning cap,

FIG. 16 shows a support structure of an atrioventricular heart valve prosthesis which is completely crimped in the valve capsule of FIG. 10,

FIGS. 17 to 22 show steps following the step of FIG. 9 for mounting the prosthesis on a delivery system for anterograde access to the native valve, in particular:

FIG. 17 shows the support structure of an atrioventricular heart valve prosthesis with the atrial crown crimped on a valve support provided on the delivery system,

FIG. 18 shows the support structure of an atrioventricular heart valve prosthesis with the atrial crown inserted in a valve capsule provided on the delivery system,

FIG. 19 shows the support structure of an atrioventricular heart valve prosthesis as in FIG. 18 and with the connecting arms 52 straightened and joined to each other,

FIG. 20 shows the support structure of an atrioventricular heart valve prosthesis with the atrial crown inserted in the valve capsule and the ventricular crown crimped by the crimper and a crimping cap,

FIG. 21 shows the crimping cap of FIG. 20 inserted on the ventricular crown of the support structure of an atrioventricular heart valve prosthesis and

FIG. 22 shows the valve capsule advanced up to the crimping cap.

### DETAILED DESCRIPTION

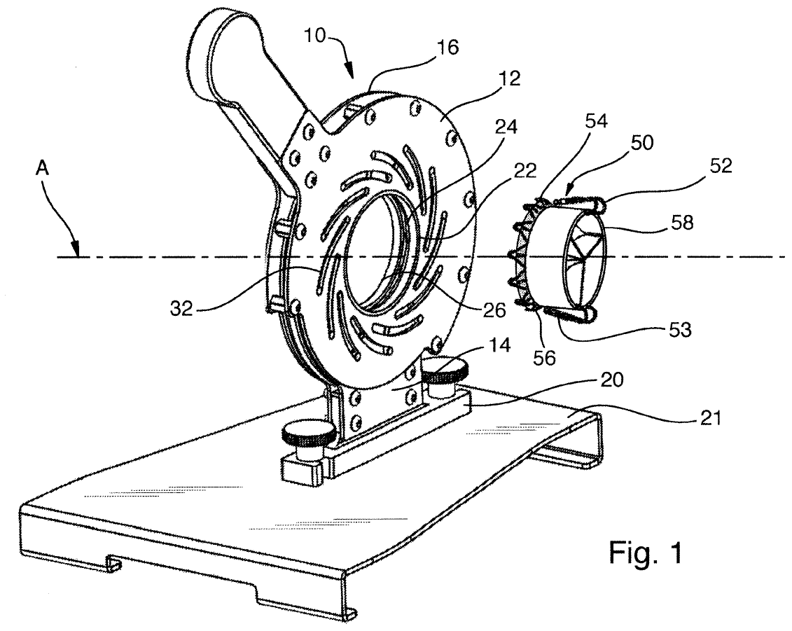

Now with reference to the drawings, FIG. 1 shows a crimper 10 which is suitable for crimping implantable devices, such as, for example, heart valve prostheses for minimally invasive implantation. One example of such a prosthetic device is described in the patent application PCT/IB2020/056960 by the same Applicant. This involves a prosthesis for an atrioventricular valve which is formed by a prosthetic structure for supporting and interfacing with the native valve and by a set of flexible prosthetic leaflets which are fixed therein. The prosthesis particularly comprises a support structure 50 (depicted in FIG. 1), that is to say, a central body which can expand radially and on which there are fixed prosthetic leaflets which, during use, functionally replace the native valve. The support structure is provided with connecting arms 52 which terminate as connection blocks 53 at an annular portion which is not illustrated. The support structure has an atrial crown 56, that is to say, the end of the support structure which during use is directed towards the atrium, that is to say, the inlet end for the blood flow, and a ventricular crown 58, that is to say, the end which during use is directed towards the ventricle, that is to say, the outlet end of the blood flow.

This support structure can be made from a super-elastic material, typically an equiatomic alloy of nickel and titanium (known commercially as Nitinol) and the crimping action is therefore directed towards deforming it so that it changes from a radially expanded condition to a radially contracted condition which is compatible with the dimensions of the delivery system and the transcatheter insertion. To do so, action is applied counter to the elastic reaction force of the structure itself, which tends to maintain the expanded configuration.

The crimper 10 preferably comprises a pair of plates 12, 16 which are connected to each other and which form a first plates system. The crimper 10 further comprises a second plates system which is preferably fixedly joined to the base of the crimper. In the embodiment depicted, the second plates system comprises a plate 14. The two plates systems 12, 16 and 14 are able to rotate with respect to each other about a common axis A. The plates of the first plates system 12, 16 are fixed to each other, with the plate 14 of the second plates system being interposed between them. The plate 14, which is only partially visible in the Figures, comprises a support 20 for fixing to a base 21 of the crimper. The plates 12, 14 and 16 have a hole which is designated 22, 24, 26, respectively. The holes 22, 24, 26 of the plates 12, 14, 16 are aligned relative to the same rotation axis A.

The first plates system 12, 16 is fixed to a handle 28. The handle 28 allows an operator to rotate the first plates system 12, 16 with respect to the plate 14.

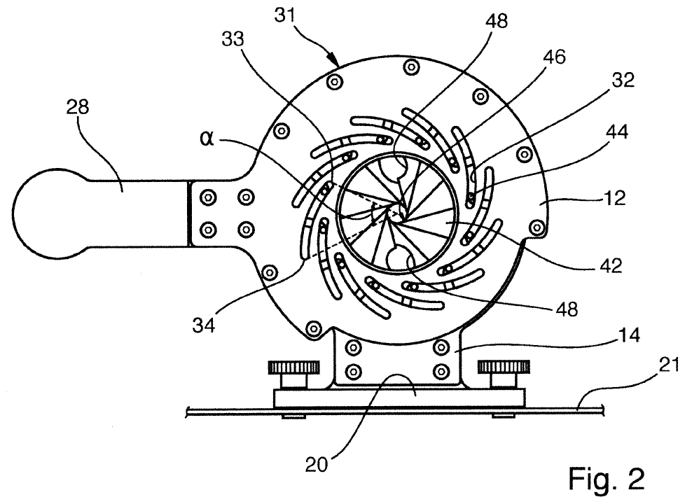

FIG. 2 shows the plate 12 of the first plates system which has curved slots 32 which preferably define circular arcs. The slots 32 are positioned radially around the hole 22 so that, for each slot 32, an internal end 33 is near the hole 22 of the plate 12 and an external end 34 is near an edge 31 of the plate 12. Furthermore, the slots 32 have the concavity always directed in the same direction (clockwise or counter-clockwise). The angular position of the internal end 33 of each slot 32 is different from the angular position of the external end 34 of the same slot and the angular distance a between the two ends of the same slot 32 is constant for all the slots 32. The slots 32 have a constant angular distance.

The plate 16 of the first plates system has slots not illustrated in the drawings, which are completely identical to and coupled with the slots 32 of the plate 12 described above, such that each slot 32 on plate 12 corresponds to a notch on plate 16.

The plate 14 has slots which are not illustrated in the drawings and which are similar to the above-described slots 32 of the plate 12 but with the concavity orientated in an opposite direction with respect to the orientation adopted for the first plates system 12, 16 so that each slot on the plate 14 intersects with at least one slot 32 on the first plates system 12, 16.

One constructive solution of the crimper provides for a plurality of crimping structures to be accommodated between the plate 12 and the plate 14, in the example in the form of wedges 42. Each of them is provided with at least one sliding element 44 which can slide in the above-described slots. The crimping structures and the slots are configured so that the crimping structures collectively define a hole 46 with a variable diameter in accordance with the mutual positioning of the crimping structure. The diameter of the hole 46 is variable by rotating the first plates system 12, 16 with respect to the plate 14, by acting on the handle 28; FIG. 2 shows the crimper with a hole 46 with a reduced diameter. In this configuration, there may also be noted the presence on the crimping structures of a pair of apertures 48, the use of which will be described below.

The hole 46 at its maximum diameter is able to receive the non-deformed support structure 50 of a prosthesis; by positioning the support structure 50 in the hole 46 and reducing the diameter thereof, therefore, it is possible to locally reduce the radial size of the support structure, focusing the crimping action into a precise portion having axial length which is limited with respect to the axial length of the prosthesis. The crimping is carried out in a plane, that is to say, the crimping action is applied with a high level of precision along a line which is determined by the intersection between the support structure 50 and a plane which is transverse thereto and which is defined by the crimping structures. However, the Applicant has found by experimentation that, with prostheses of certain geometries and dimensions, the crimping carried out only with a crimper of the above-described type is not effective and may cause drawbacks, such as the collapse or the irregular deformation of the support structure which can also introduce risks for the integrity of the prosthesis itself, in addition to making the crimping itself difficult or even impossible.

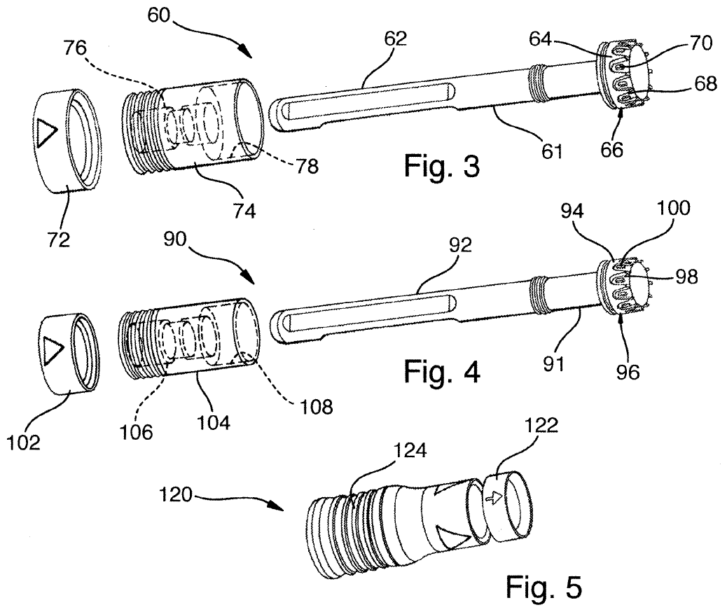

In order to avoid the above-described risks, the Applicant has developed the set of instruments shown in FIGS. 3, 4 and 5. They will be referred to below as the first pair of instruments 60, second pair of instruments 90 and third instrument 120, respectively.

The first pair of instruments 60 comprises an instrument 61 and a crimping ring 72. The instrument 61 comprises a handle 62 which terminates at one end with a cylindrical body 64, which is preferably but not necessarily coaxial with the handle, on the lateral surface 66 of which there are provided seats 68 for receiving therein the atrial crown 56 of the support structure. In the specific case, as can be seen in FIG. 1, the support structure has in the atrial crown a plurality of loops 54 which rest around projections 70 which are present in the seats 68. In case the instrument 61 is to be used with a support structure with a different shape, naturally there will be seats 68 which are shaped differently, coherently with the geometry of an end of the support structure. Not necessarily each seat 68 has to provide a projection 70. With the constructive solution of the valve prosthesis shown in FIG. 1, it is preferable for at least one of the seats 68 to have a projection 70 so as to apply an anchoring action of the support structure to the cylindrical body 64. With different geometries of the support structure, it is possible that this anchoring action is applied by the seat 68 itself. There is associated with the cylindrical body 64 an atrial cap 74. The atrial cap 74 is a structure which is generally tubular and which comprises a hole 76 for allowing the insertion and sliding over the handle 62. It further comprises a cavity 78 which is shaped and sized to receive therein the cylindrical body 64 and to constrain the end of the support structure inside the seats 68, counteracting the elastic radial force which is exerted by the structure itself. The atrial cap is preferably made of transparent material, for example, plastics material, in order to make the support structure inside the seats 68 visible to the operator.

With reference to the valve prosthesis 50 which is shown in FIG. 1, the cylindrical body 64 of the instrument 61 has a diameter which is: smaller than the atrial crown 56 in a freely expanded configuration, that is to say, before being crimped.

The first pair of instruments 60 further comprises, as mentioned, a crimping ring 72. With reference to the valve prosthesis 50 shown in FIG. 1, the internal diameter of the crimping ring 72 is smaller than the ventricular crown 58 in a freely expanded configuration, that is to say, before being subjected to the crimping procedure. Preferably, the atrial cap 74 has to have an external dimension which allows the crimping ring 72 to slide thereon.

In the event that it is advantageous, for the crimping procedure, to have a crimping ring 72 with an internal diameter smaller than the external dimension of the atrial cap, the crimping ring 72 can be constructed in an open form or even subdivided into at least two separate pieces with an additional component which is able to encircle and to stabilize the ring at the time it is coupled to the ventricular portion of the support structure.

The second pair of instruments 90 is generally analogous to the first pair of instruments 60 described above but has components with dimensions for the crimping of the support structure with a diameter smaller than the first pair of instruments 60. It comprises an instrument 91 and a crimping ring 102. The instrument 91 has a handle 92 which terminates at one end with a cylindrical body 94 having an external diameter smaller than the cylindrical body 64. On the lateral surface 96 of the cylindrical body 94 there are provided seats 98 for receiving therein one end of the support structure; in the case of the support structure which is depicted, the seats 98 are formed so as to receive therein the ends of the loops of the atrial crown 56. The seats are nearer to each other than the seats 68, having dimensions so as to receive the support structure 50 in a more crimped configuration, that is to say, with a smaller radial dimension. Projections 100 can still be present in at least one of the seats 98 in accordance with the geometry of the end of the support structure. In accordance with the geometry of the end of the support structure, in fact, in order to fixedly join the cylindrical body 94 to the support structure, the geometry of the seats 98 may be sufficient or additional anchoring elements may be necessary. There is associated with the cylindrical body 94 an atrial cap 104 which comprises a hole 106 for allowing insertion and sliding over the handle 92. The atrial cap 104 further comprises a cavity 108 which is formed and sized so as to receive the cylindrical body 94 therein. The cavity 108 is further formed to secure the end of the support structure inside the seats 98, counteracting the elastic radial force exerted by the structure itself.

The second pair of instruments 90 further comprises a crimping ring 102. The internal diameter of the crimping ring 102 is preferably smaller than the internal diameter of the crimping ring 72 of the first pair of instruments 60. Preferably, the atrial cap 104 has an external dimension which allows the crimping ring 102 to slide thereon.

The third pair of instruments 120 comprises a crimping ring 122 and a positioning cap 124. The crimping ring 122 has an internal diameter which is preferably smaller than the internal diameter of the crimping ring 102 of the second pair of instruments 90. The positioning cap 124 is characterized by a dead-end internal cavity.

The three pairs of instruments described above are provided for use together with a crimper, preferably with the crimper 10, for a greater versatility and efficiency when crimping support structures of various lengths and with variable cross-sections which are not necessarily cylindrical. To this end, the crimping is carried out through successive steps, preferably by partially crimping the atrial portion and the ventricular portion in turn, until achieving a dimension which is compatible with the mounting of the implantable prosthesis in a delivery system. There will now be described the crimping procedure.

With non-limiting reference to the valve prosthesis 50 described in FIG. 1, if it is made from Nitinol, before crimping the support structure 50 it is advantageous to immerse it in a physiological solution at a temperature lower than the transformation temperature thereof. There is thereby obtained a phase transformation of the alloy, from austenitic to martensitic, which makes the support structure plastically deformable and therefore easier to crimp. The same operation can be carried out several times in the course of the steps described below.

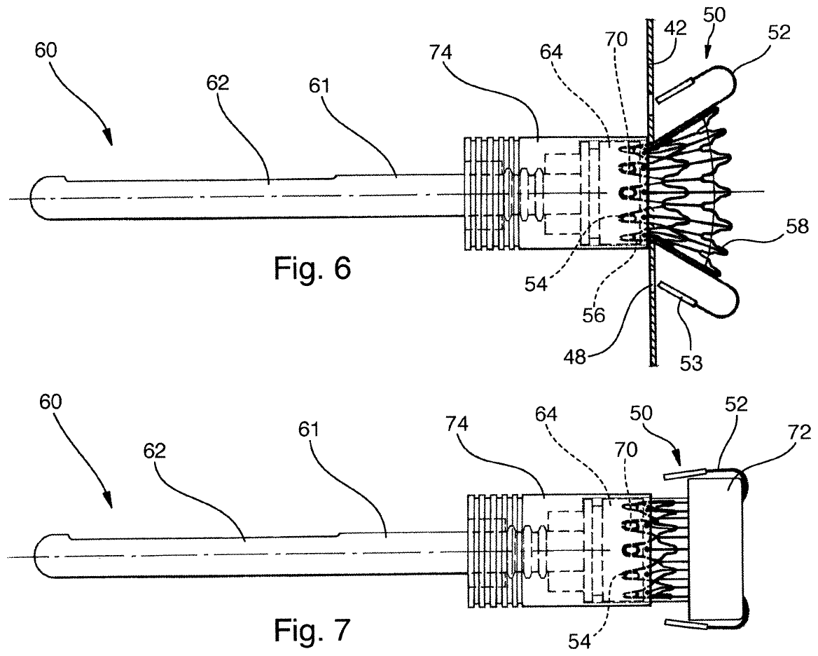

In a first step of the crimping procedure, the handle 28 of the crimper 10 is rotated until the hole 46 is completely opened. Then, one end of the support structure is inserted in the hole 46 of the crimper 10, positioning the connecting arms on one side of the crimper 10 (on the right, in the example in FIG. 6) and the atrial crown 56 on the other side. The handle 28 is then rotated in the opposite direction until the wedges 42 are in contact with the base of the atrial crown 56 and the support structure is stable inside the hole 46 of the crimper 10.

Then, the cylindrical body 64 of the instrument 61 is inserted inside the support structure, positioning it so that the atrial loops 54 of the support structure are aligned with the seats 68 created on the external surface 66. Then, the atrial crown 56 of the support structure is crimped until each of the atrial loops 54 fits into the respective seat 68 (FIG. 6) and around the respective projection 70, where this is present. The atrial cap 74 is then slid on the cylindrical body 64 and on the atrial crown 56 of the support structure until it comes into contact with the wedges 42 of the crimper 10. The atrial portion of the support structure is thereby stably anchored on the instrument 61, constrained by the atrial cap 74 at a predetermined diameter. The diameter is equal to the radial dimension of the cavity 78, preferably smaller than the initial diameter of the support structure.

The crimper 10 is then opened and the support structure is removed from it. By maintaining the support structure clamped to the instrument 61, as shown in FIG. 7, the connecting arms 52 are slightly spaced apart and the crimping ring 72 is slid on the atrial cap 74 and on the ventricular portion of the support structure until reaching the opposite end, thereby obtaining a corresponding radial reduction of the ventricular crown.

In a second step of the crimping procedure, the support structure is inserted again, still mounted on the instrument 61 and with the crimping ring 72 positioned on the ventricular crown, in the open hole 46 of the crimper. Again, the handle 28 of the crimper 10 is actuated in order to adapt the hole 46 to the diameter of the support structure; then, the atrial cap 74 is retracted, releasing the atrial crown 56. Then, the instrument 61 is disengaged from the support structure, the atrial crown 56 of which remains stable inside the crimper 10 and at the diameter fixed by it.

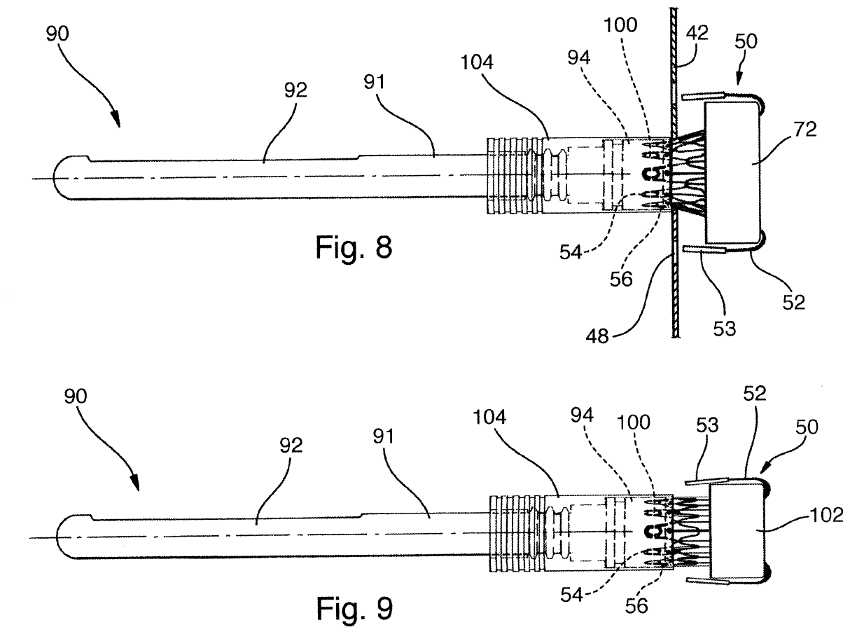

Then, the cylindrical body 94 of the instrument 91 is inserted inside the atrial crown 56 of the support structure in place of the removed instrument 61. As previously described for the instrument 61, the atrial crown 56 of the Support structure is further crimped until each of the atrial loops 54 is received in the respective seat 98 (FIG. 8) and around the projection 100 where this is present. The atrial cap 104 is then slid on the cylindrical body 94 and on the atrial crown 56 of the support structure. The atrial portion of the support structure is thereby stably anchored on the instrument 91 and is constrained thereto by the atrial cap 104 at a predetermined diameter. The diameter of the atrial portion is particularly determined by the radial dimension of the cavity 108. This diameter is smaller than the diameter which it had in the step of FIG. 6, in which the atrial portion was secured to the instrument 61 by the atrial cap 74 and therefore the diameter was determined by the radial dimension of the cavity 78 thereof.

The crimper 10 is then opened and the support structure is removed from it. By maintaining the support structure secured to the instrument 91, the crimping ring 72 of the first pair of instruments 60, still positioned on the ventricular end of the support structure, is removed. The connecting arms 52 are slightly spaced apart and the crimping ring 102 is slid on the atrial cap 104 and on the ventricular portion of the support structure until reaching the opposite end thereof, thereby obtaining a further radial reduction of the ventricular crown. The support structure which is configured in this manner is shown in FIG. 9. The support structure can be maintained for a relatively long time in this configuration because it is balanced in terms of the value of the reduction of the diameter along the axial development of the structure.

With the support structure still mounted on the instrument 91, it is possible to orientate the prosthesis in a predetermined direction, in case the prosthesis should have to be coupled with the delivery system while complying with a predetermined orientation. By maintaining this orientation, the support structure is inserted, still mounted on the instrument 91 and with the crimping ring 102 positioned at the ventricular end, in the hole 46 of the crimper. Again, the handle 28 of the crimper 10 is actuated in order to narrow the hole 46 to the diameter of the support structure; then, the atrial cap 104 is removed, releasing the atrial crown 56 and disengaging the instrument 91 from the support structure.

It is possible to provide, in the crimping procedure, additional intermediate steps which are similar to the ones described until now in order to achieve a size of the support structure and therefore of the prosthesis which is compatible with the final mounting step on the delivery system.

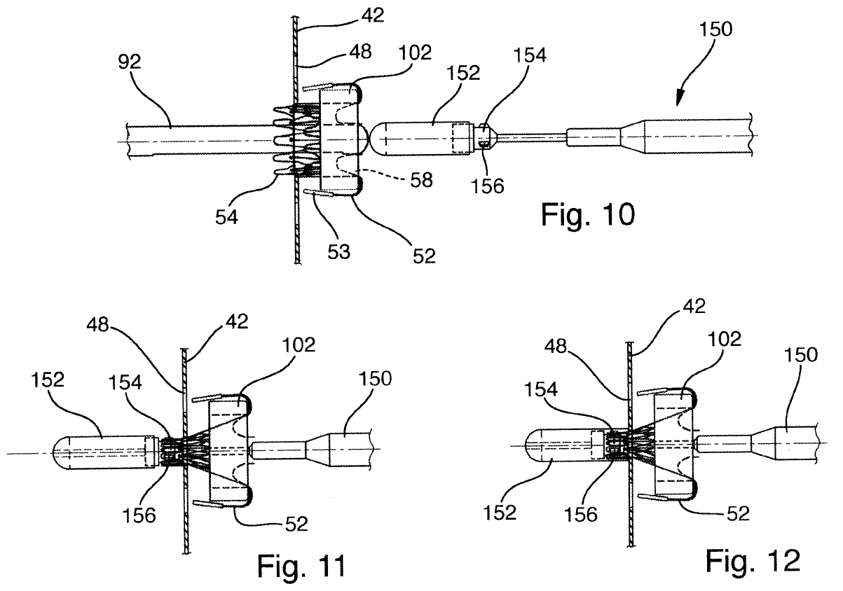

The final step of the crimping procedure involves mounting the implantable prosthesis on the delivery system. FIG. 10 shows a delivery system 150 of the valve, for example, of the type described in the patent IT0001414559 by the same Applicant, which may comprise a valve capsule 152, in which the implantable prosthesis has to be inserted, or also only a portion thereof, in order to allow the release thereof in the desired location. The delivery system 150 is provided for retrograde access to the valve. The support structure, when it is inserted in the capsule, can be anchored around a valve support 154, suitably shaped.

In order to carry out the mounting of the prosthesis on the delivery system 150, the valve capsule 152 is inserted through the prosthesis 50 until positioning the support 154 in the region of the atrial loops 54 of the support structure so that the capsule 152 initially covers the atrial portion of the prosthesis and is then advanced onto the ventricular portion. The release of the implantable prosthesis during the implantation thereof occurs according to the reverse sequence. The design of the delivery system 150 is in fact intended to initially carry out the release of the ventricular portion of the support structure, maintaining the atrial portion of the prosthesis 50 still inside the capsule 152 and anchored to the valve support 154. The final release of the atrial portion as well, which occurs by completely disengaging the capsule 120 from the support 154, performs the final implantation of the prosthesis.

In order to prevent the advancement of the capsule 152 through the prosthesis from causing damage to the prosthetic leaflets which are located inside the support structure, it is possible to use the handle 62 of the instrument 61 or the handle 92 of the instrument 91 in order to separate and open the prosthetic leaflets, allowing safe passage of the valve capsule 120. To this end, it is possible to insert the handle 92 or 62 through the prosthesis, by introducing it from the side of the atrial crown 56, that is to say, from the left in the Figure, in order to keep the prosthetic leaflets open. From the opposite side (from the right in the Figure), the tip of the valve capsule 152 is approached. Then, the tip of the capsule is placed against the handle 92 of the second instrument. By maintaining the two components in contact, the capsule of the valve is advanced through the support structure without damaging the prosthetic leaflets.

While taking care, if required by the crimping or implantation procedure, to orientate the delivery system 150 with respect to the prosthesis 50, the atrial crown 56 is then further crimped on the valve support 154 (FIG. 11). In this step, it is advantageous to prevent the atrial loops from overlapping, maintaining them substantially equidistant. In order to anchor the support structure, and in particular the atrial loops 54, on the delivery system by means of the valve support 154, on the valve support 154 there are preferably provided one or more projections 156 which are able to be engaged in the atrial loops.

When the atrial crown 56 is correctly positioned on the valve support 154, the valve capsule 152 is moved so as to cover the atrial crown 56 of the valve, as shown in FIG. 12. Then, the support structure and the delivery system of the valve are removed from the crimper and it is verified that the atrial crown 56 of the support structure is correctly positioned inside the valve capsule 152.

It may be noted that the final stage of crimping of the valve, as described below, is preferably carried out shortly before the use of the delivery system for the implantation procedure. In fact, it is the operation which completes the crimping of the ventricular portion of the prosthesis, with the consequent maximum compaction of the biological component of the prosthesis, and in particular of the prosthetic leaflets. A long stay of the prosthetic leaflets in a compact configuration may lead to their partial drying out, and in any case cause their damage.

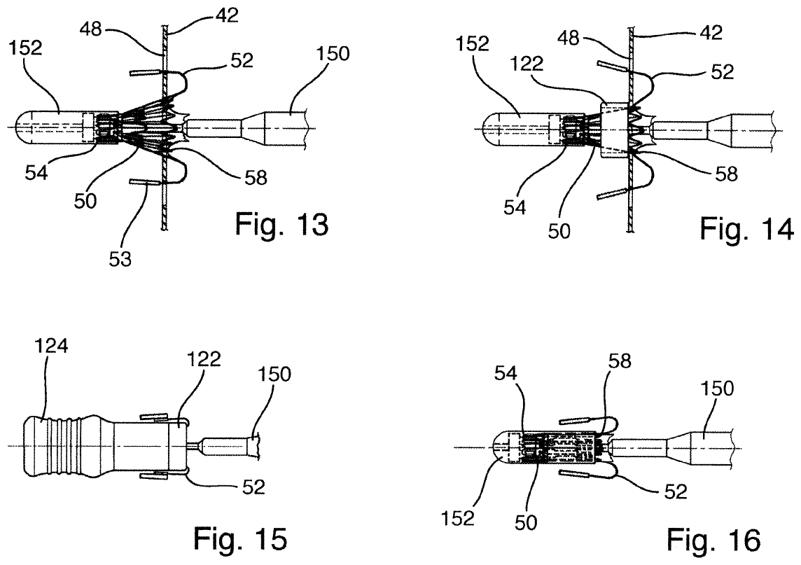

In order to carry out the final crimping of the support structure, the crimping ring 102 of the second pair of instruments 90 is removed from the support structure. With reference to FIG. 13, the support structure is then introduced into the hole 46 of the crimper 10, in such a position that the wedges 42 of the crimper 10 are positioned in correspondence with the ventricular crown 58 of the support structure. The support structure of the atrioventricular valve prosthesis 50 taken as reference for the description of the crimping procedure and depicted here has, as seen previously, a pair of connecting arms 52 which could interfere with the correct crimping of the ventricular portion. In order to overcome this problem, which is strictly linked with this specific configuration of the support structure, but not necessarily present in the case of a support structure which is shaped differently, the connecting arms 52 are introduced into the apertures 48 provided in the wedges 42.

Then, the crimper 10 is actuated, reducing the hole 46 to a diameter which allows the introduction, from the side of the valve capsule 152, of the crimping ring 122 of the third instrument 120. The crimping ring 122 is pushed up to the wedges 42 of the crimper. It is also possible to use the positioning cap 124 in order to more easily push the crimping ring 122 towards the wedges 42.

The crimper is opened and the group formed in this manner is extracted. Then, the positioning cap 124 of the third instrument 120 is slipped over the valve capsule 152 and the positioning cap is pressed so that the crimping ring 122 reaches the ventricular end of the support structure, as shown in FIG. 15. The depth of the cavity present in the positioning cap 124 can be appropriately sized so that the tip of the capsule 152 comes into contact with the bottom of the cavity exactly in correspondence with the correct positioning of the crimping ring 122 at the ventricular end of the support structure. In this manner, the ring 122 is prevented from slipping off the support structure by mistake. Finally, the positioning cap 124 is removed.

Having substantially stabilized the support structure in the configuration of minimal spatial requirement by means of the valve capsule 152 at the atrial side and with the crimping ring 122 at the ventricular side, the capsule 152 is advanced along the whole remaining length of the support structure. In order to complete this final step, it may be advantageous to further use the crimper 10 in order to reduce the radial section of the implantable prosthesis in the immediate vicinity of the edge of the valve capsule 152. In this manner, it is possible to prevent the jamming of the capsule itself, as well as the irregular arrangement of the components of the prosthesis in the configuration of minimal spatial requirement.

The support structure 50 is thereby crimped and inserted in the valve capsule 152.

It may be noted that the procedure described above may naturally vary in accordance with the specific features of the support structure. An important characteristic of the procedure is that the support structure is not crimped at the same time over the entire length thereof, but this is carried out first by crimping the atrial crown 56 and then the ventricular crown 58, possibly passing through intermediate stable configurations, in order to avoid any risk of collapse of or damage to the prosthesis and with the advantage of making the procedure easier to carry out. Furthermore, the possibility of acting in the reverse manner, by first crimping the ventricular crown and then the atrial crown, is not excluded as a matter of principle. It is further possible to use a crimper which is shaped differently, as long as it allows crimping of the support structure along a crimping plane which is directed perpendicularly to the axis thereof.

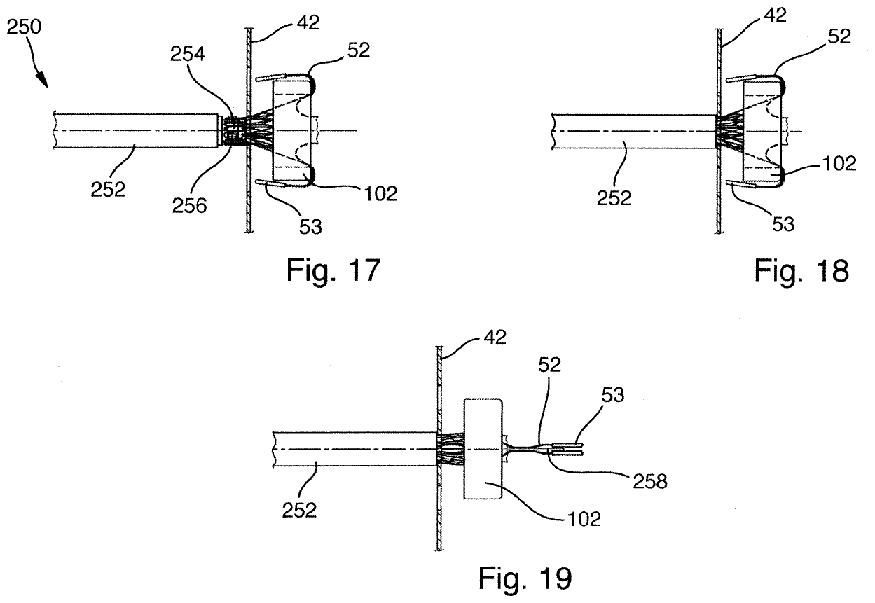

An additional example of the procedure is described below with reference to FIGS. 17 to 22 which show steps following the step of FIG. 9 for fixing the support structure on a delivery system for anterograde access to the native valve. A delivery system for anterograde access to the mitral valve is described, for example, in the International Patent Application WO 2021/014400 by the same Applicant and the subsequent Italian Patent Application IT102022000001166.

The delivery system 250 for a procedure with an anterograde approach, which is partially visible in FIG. 17, comprises a capsule 252 and a valve support 254. There are preferably provided on the valve support 254 one or more projections 256 which are suitable for engaging in the atrial loops 54 in order to anchor the support structure and particularly the atrial crown to the delivery system.

After carrying out the steps described above up to FIG. 9, the support structure is inserted, still mounted on the instrument 91 and with the crimping ring 102 positioned at the ventricular end, in the hole 46 of the crimper. With the support structure still mounted on the instrument 91, it is possible to orientate the prosthesis in a predetermined direction in case the prosthesis has to be coupled with the delivery system while complying with a predetermined orientation. By maintaining this orientation, the handle 28 of the crimper 10 is actuated in order to narrow the hole 46 to the diameter of the support structure; the atrial cap 104 is then removed, releasing the atrial crown 56 and disengaging the instrument 91 from the support structure.

Then (FIG. 17), the valve support 254 is inserted inside the atrial crown 56. Then, the atrial crown is crimped until it engages on the projections 256 of the valve support 254.

Subsequently, the valve capsule 252 is advanced until it blocks the atrial crown in a stable manner on the valve support 254, as shown in FIG. 18.

In order to be able to use a capsule with a reduced radial dimension, as described in the prior Italian Patent Application IT102022000001166, the connecting arms 52 of the prosthesis are then straightened (FIG. 19) the connection blocks 53 are joined to each other, for example by means of a guide wire 258 which passes through special appendages which are provided on the blocks themselves.

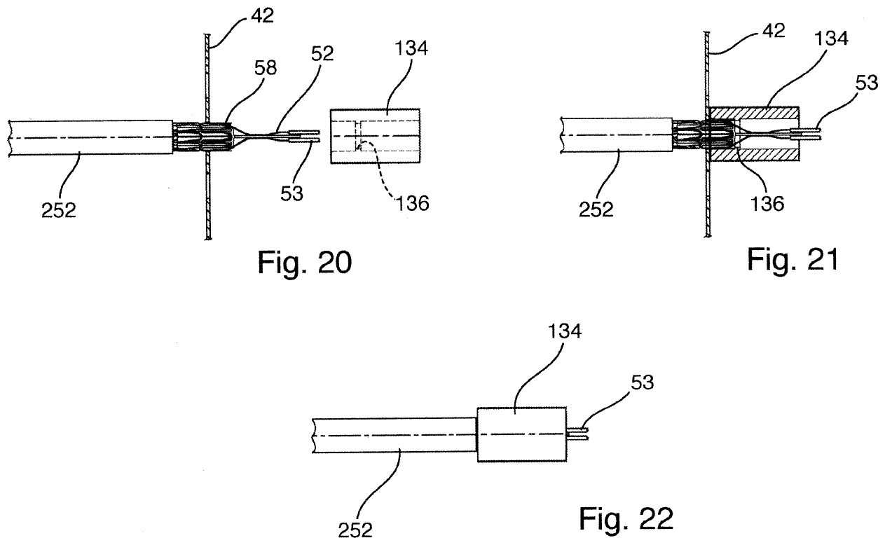

With reference to FIG. 20, in case of a delivery system for an anterograde procedure, in place of the instruments of FIG. 5 it is possible to use a ventricular cap 134. The ventricular cap 134 has a tubular shape with an internal diameter equal to the desired diameter of the crimped support structure. Inside, the ventricular cap 134 has an abutment element 136.

In order to carry out the final crimping of the support structure, the crimper 10 is slightly opened and moved into such a position that the wedges 42 of the crimper 10 are in correspondence with the ventricular crown 58 of the support structure; in order to do this, the crimping ring 102 of the second pair of instruments 90 is moved slightly. Then, the ventricular portion is crimped to a diameter which is compatible with the internal diameter of the ventricular cap. Then, the crimping ring 102 is removed from the ventricular side of the support structure, as shown in FIG. 20.

Then, from the same side of the support structure, the ventricular cap 134 is inserted until having the ventricular end 58 of the support structure in contact with the abutment element 136 inside the ventricular cap 134, as can be seen in FIG. 21.

The support structure is thereby stable at the crimping diameter between the valve capsule for the atrial crown and the ventricular cap 134. At this point (FIG. 22), the crimping of the valve is completed by advancing the capsule 252 over the crimped support structure up to the ventricular cap, when needed with the assistance of the crimper 10 which possibly can be subsequently moved to sections immediately distal with respect to the edge of the valve capsule, to facilitate the advancement of the capsule 252.

Naturally, the principle of the invention remaining the same, the forms of embodiment and details of construction may be varied widely with respect to those described and illustrated without thereby departing from the scope of the invention. In accordance with the characteristics of the support structure, for example, the possibility of providing a set composed only of one of the first and second pairs of instruments 60 and 90 should not be excluded, preferably in conjunction with a third instrument of the type of the instrument 120, for a crimping procedure which is more rapid but less gradual, or conversely a set with three or more pairs of instruments of the type of the pairs 60 and 90, with a diameter which gradually decreases, for a crimping procedure which is even more gradual, preferably in conjunction with a third instrument of the type of the instrument 120.

### Embodiments

1. A procedure for crimping a support structure (50) of an implantable device, the support structure having two ends, wherein the procedure comprises the steps of:
a. crimping one of the two ends of the support structure,
b. subsequently crimping the other end.
2. A procedure according to claim 1, wherein the steps (a) and (b) are repeated at least a second time, preferably in the same order.
3. A procedure according to the preceding claim, wherein the steps (a) and (b) are repeated a third time.
4. A procedure according to any one of the preceding claims, wherein the step (a) is carried out with a crimper (10) and an instrument comprising a cylindrical body (64, 94, 154, 254) having projections (70, 100, 156, 256) at an external lateral surface thereof.
5. A procedure according to any one of the preceding claims, wherein the step (b): is carried out with an instrument comprising a crimping ring (72, 102, 122) or a ventricular cap (134).
6. A procedure according to any one of the preceding claims, wherein the implantable device is an atrioventricular heart valve and the two ends are an atrial crown and a ventricular crown, respectively.
7. A set of instruments for crimping a support structure (50) of an atrioventricular heart valve, comprising an atrial crown (56) and a ventricular crown (58), comprising
   at least one instrument (61, 91) comprising a handle (62, 92) which terminates at one end as a cylindrical body (64, 94) on the lateral surface of which there are provided seats (68, 98) for receiving therein the atrial crown (56) of the support structure, and an atrial cap (74, 104) which can slide on the cylindrical body (64, 94) in order to block the atrial crown there,
   at least one crimping ring (72, 102) for crimping the ventricular crown (58).
8. A set of instruments according to the preceding claim, further comprising an additional crimping ring (122) for further crimping the ventricular crown and a positioning cap (124) in order to assist in an insertion operation of the additional crimping ring (122) on the ventricular crown of the support structure (50).
9. A set of instruments according to claim 7, further comprising a ventricular cap (134) for further crimping the ventricular crown in order to assist in an insertion operation of the support structure (50) on a delivery system (250).
10. A procedure for crimping a support structure of an atrioventricular heart valve prosthesis according to any one of claims 1 to 6, wherein there is used a set of instruments for crimping according to claim 7, 8 or 9 and a crimper.
11. A crimper comprising at least a first plate (12, 16) and at least a second plate (14) which are able to rotate with respect to each other about a common axis (A), wherein there are received between the first plate (12, 16) and the second plate (14) a plurality of crimping structures which are configured so as to collectively define a hole (46) with a variable diameter in accordance with the mutual positioning of the crimping structures, the crimping structures further defining a pair of apertures (48).

1. A set of instruments for crimping a support structure of a heart valve comprising a first crown and a second crown, comprising:
at least one instrument comprising a handle terminating at one end as a cylindrical body, the cylindrical body having a lateral surface and seats on the lateral surface for receiving therein the first crown of the support structure, and a cap slidable on the cylindrical body to block the first crown on the cylindrical body; and
at least one crimping ring for crimping the second crown.

2. The set of instruments according to claim 1, further comprising:
a second instrument having a cylindrical body and a cap, diameters of the cylindrical body and the cap being smaller than respective diameters of the cylindrical body and the cap of the at least one instrument and,
a second crimping ring having a diameter smaller than a diameter of the at least one crimping ring.

3. The set of instruments according to claim 1, further comprising an additional crimping ring for further crimping the second crown and a positioning cap to assist in an insertion operation of the additional crimping ring on the second crown of the support structure.

4. The set of instruments according to claim 1, further comprising a cap for further crimping the second crown to assist in an insertion operation of the support structure on a delivery system.

5. The set of instruments according to claim 1, wherein the heart valve is an atrioventricular heart valve and the first and second crowns are an atrial crown and a ventricular crown, respectively.

6. A procedure for crimping, with a set of instruments according to claim 1, a support structure of a heart valve, the support structure having a first crown and a second crown, wherein the procedure comprises the steps of:
a. crimping the first crown of the support structure around the cylindrical body of the at least one instrument and fixing the first crown to the cylindrical body with a cap; and,
b. subsequently crimping the second crown with the at least one crimping ring.

7. The procedure according to claim 6, further comprising the steps of:
c. further crimping the first crown around a cylindrical body of a delivery system of the heart valve, the cylindrical body having projections on an external lateral surface thereof; and
d. subsequently crimping the second crown.

8. The procedure according to claim 7, wherein the step (d) is carried out with an instrument comprising a crimping ring or a cap.

9. The procedure according to claim 7, further comprising, between the step (b) and step (c), the steps of:
b1. further crimping the first crown of the support structure around a cylindrical body having a diameter smaller than the diameter of the cylindrical body used in the step (a) and greater than the diameter of the cylindrical body used in the step (c);
b2. crimping the second crown with a crimping ring with a diameter smaller than the crimping ring used in the step (b).

10. The procedure according to claim 7, wherein the step (a) and/or the step (b1) and/or the step (c) and/or the step (d) are carried out with a crimper.

11. A procedure for crimping a support structure of a heart valve, the support structure having a first crown and a second crown, wherein the procedure comprises the steps of:
i. crimping the first crown of the support structure to a first diameter of the first crown;
ii. maintaining the first crown crimped at the first diameter of the first crown, crimping the second crown to a first diameter of the second crown;
iii. maintaining the second crown crimped at the first diameter of the second crown, crimping the first crown to a second diameter of the first crown smaller than the first diameter of the first crown; and
iv. maintaining the first crown crimped at the second diameter of the first crown, crimping the second crown at a second diameter of the second crown smaller than the first diameter of the second crown.

12. The procedure according to claim 11, further comprising the steps of:
v. maintaining the second crown crimped at the second diameter of the second crown, crimping the first crown at a third diameter of the first crown smaller than the second diameter of the first crown; and
vi. maintaining the first crown crimped at the third diameter of the first crown, crimping the second crown at a third diameter of the second crown smaller than the second diameter of the second crown.

13. The procedure according to claim 12, wherein the step (i) and/or the step (iii) and/or the step (v) and/or the step (vi) are carried out with a crimper and an instrument comprising a cylindrical body having projections on an external lateral surface thereof.

14. The procedure according to claim 11, wherein the heart valve is an atrioventricular heart valve and the first and second crowns are an atrial crown and a ventricular crown, respectively.

15. A crimper comprising at least a first plate and at least a second plate able to rotate with respect to each other about a common axis, a plurality of crimping structures in the form of wedges and received between the first plate and the second plate, the plurality of crimping structures being configured so as to collectively define a hole with a variable diameter in accordance with the mutual positioning of the crimping structures, the crimping structures further defining a pair of apertures.
